(19) United States
(12) Patent Application Publication
(10) Pub. No.: US 2006/0199803 A1
(43) Pub. Date: Sep. 7, 2006

(54) Compounds

(75) Inventors: Marc Geoffrey Hummersone, Cambridge (GB); Sylvie Gomez, Cambridge (GB); Keith Allan Menear, Cambridge (GB); Xiao-Ling Fan Cockcroft, Cambridge (GB); Peter Edwards, Cambridge (GB); Ming-lai Loh, Cambridge (GB); Graeme Cameron Murray Smith, Cambridge (GB)

Correspondence Address: MICHAEL BEST & FRIEDRICH, LLP, ONE SOUTH PINCKNEY STREET, P O BOX 1806, MADISON, WI, 53701, US

(73) Assignee: Kudos Pharmaceuticals Ltd, Cambridge (GB)

(21) Appl. No.: 11/361,213

(22) Filed: Feb. 24, 2006

Related U.S. Application Data
(60) Provisional application No. 60/656,178, filed on Feb. 25, 2005.
(60) Provisional application No. 60/742,403, filed on Dec. 5, 2005.

(30) Foreign Application Priority Data
Feb. 25, 2005 (GB) 0503961.5

(51) Int. Cl. A61K 31/551; A61K 31/541; A61K 31/519; A61K 31/5377; C07D 487/02

(52) U.S. Cl. 514/218; 514/234.2; 514/227.8; 514/264.11; 540/575; 544/60; 544/114; 544/279

(57) ABSTRACT

Compounds of formula l: 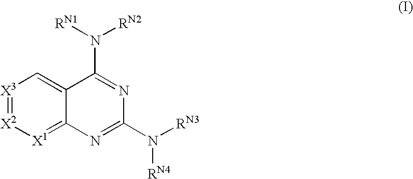
and isomers, salts, solvates, chemically protected forms, and prodrugs thereof one of X¹, X² and X³ is N, and the others are CH; R^{N1} and R^{N2} together with the nitrogen atom to which they are attached form a nitrogen-containing heterocyclic ring having from 4 to 8 ring atoms; R^{N3} and R^{N4} together with the nitrogen atom to which they are attached form a nitrogen-containing heterocyclic ring having from 4 to 8 ring atoms and their use in treating diseases ameliorated by the inhibition of mTOR.

### CROSS-REFERENCE TO RELATED APPLICATIONS

This application claims the priority benefit of U.S. Provisional Patent Application Ser. No. 60/656,178 filed on Feb. 25, 2005, U.S. Provisional Patent Application Ser. No. 60/742,403 filed on Dec. 5, 2005 and United Kingdom Patent Application No, 0503961.5 filed Feb. 25, 2005. These applications are incorporated herein by reference.

### FIELD

The present invention relates to compounds which act as mTOR inhibitors, their use and their synthesis.

### BACKGROUND

Growth factor/mitogenic activation of the phosphatidylinositol 3-kinase (Pl3K)/AKT signalling pathway ultimately leads to the key cell cycle and growth control regulator mTOR, the mammalian target of rapamycin (alternatively referred to as FRAP (FKBP12 and rapamycin associated protein), RAFT1 (rapamycin and FKBP12 target 1), RAPT1 (rapamycin target 1)—all derived from the interaction with the FK-506-binding protein FKBP12, and SEP (sirolimus effector protein)). mTOR is a mammalian serine/threonine kinase of approximately 289 kDa in size and a member of the evolutionary conserved eukaryotic TOR kinases (refs. 1-4). The mTOR protein is a member of the Pl3-kinase like kinase (PlKK) family of proteins due to its C-terminal homology (catalytic domain) with Pl3-kinase and the other family members, e.g. DNA-PKcs (DNA dependent protein kinase), ATM (Ataxia-telangiectasia mutated). In addition to a catalytic domain in the C-terminus, mTOR contains a FKBP12/rapamycin complex binding domain (FRB). At the N-terminus up to 20 HEAT (Huntingtin, EF3, alpha regulatory subunit of PP2A and TOR) motifs are found whilst more C-terminal is a FAT (FRAP-ATM-TRRAP) domain, and at the extreme C-terminus of the protein an additional FAT domain is found (FAT-C) (refs. 5,6).

TOR has been identified as a central regulator of both cell growth (size) and proliferation, which is in part governed by translation initiation. TOR dependant phosphorylation of S6-kinase (S6K1) allows translation of ribosomal proteins involved in cell cycle progression (refs. 7-9).Cap-dependant translation is regulated by the phosphorylation of the eukaryotic translation initiation factor4E (eIF4E)-binding protein 1 (4E-BP1 (PHAS-1)). This modification prevents PHAS-1 binding eIF4E, thereby permitting formation of an active eIF4F translation complex (reviewed in refs. 10, 11, 12). Activation of these signalling elements is dependant on insulin, other growth factors and nutrients suggesting a gatekeeper role for mTOR in the control of cell cycle progression only under favourable environmental conditions. The Pl3K/AKT signalling cascade lies upstream of mTOR and this has been shown to be deregulated in certain cancers and results in growth factor independent activation in, for example, PTEN deficient cells. mTOR lies at the axis of control for this pathway and inhibitors of this kinase (e.g. sirolimus (rapamycin or Rapamune™) and everolimus (RAD001 or Certican™)) are already approved for immunosuppression and drug eluting stents (reviewed in refs. 13, 14), and are now receiving particular interest as novel agents for cancer treatment.

Tumour cell growth arises from the deregulation of normal growth control mechanisms such as the loss of tumour suppressor function(s). One such tumour suppressor is the phosphatase and tensin homologue deleted from chromosome ten (PTEN). This gene, also known as mutated in multiple advanced cancers (MMAC), has been shown to play a significant role in cell cycle arrest and is the most highly mutated tumour suppressor after p53. Up to 30% of glioblastoma, endometrial and prostate cancers have somatic mutations or deletions of this locus (refs. 15,16).

Pl3K converts phosphatidylinositol 4,5, bisphosphate (PlP2) to phosphatidylinositol 3,4,5, triphosphate (PlP3) whilst PTEN is responsible for removing the 3′ phosphate from PlP3 producing PlP2. Pl3-K and PTEN act to maintain an appropriate level of PlP3 which recruits and thus activates AKT (also known as PKB) and the downstream signalling cascade that is then initiated. In the absence of PTEN, there is inappropriate regulation of this cascade, AKT becomes effectively constitutively activated and cell growth is deregulated. An alternative mechanism for the deregulation of this cell signalling process is the recent identification of a mutant form of the Pl3K isoform, p110alpha (ref. 17). The apparent increased activity of this mutant is thought to result in increased PlP3 production, presumably in excess of that which the function of PTEN can counteract. Increased signalling from Pl3K, thus results in increased signalling to mTOR and consequently, its downstream activators.

In addition to the evidence linking mTOR with cell cycle regulation (from G1 to S-phase) and that inhibition of mTOR results in inhibition of these regulatory events it has been shown that down regulation of mTOR activity results in cell growth inhibition (Reviewed in refs. 7,18,19). The known inhibitor of mTOR, rapamycin, potently inhibits proliferation or growth of cells derived from a range of tissue types such as smooth muscle, T-cells as well as cells derived from a diverse range of tumour types including rhabdomyosarcoma, neuroblastoma, glioblastoma and medulloblastoma, small cell lung cancer, osteosarcoma, pancreatic carcinoma and breast and prostate carcinoma (reviewed in ref. 20). Rapamycin has been approved and is in clinical use as an immunosuppressant, its prevention of organ rejection being successful and with fewer side effects than previous therapies (refs. 20, 21). Inhibition of mTOR by rapamycin and its analogues (RAD001, CCI-779) is brought about by the prior interaction of the drug with the FK506 binding protein, FKBP12. Subsequently, the complex of FKBP12/rapamycin then binds to the FRB domain of mTOR and inhibits the downstream signalling from mTOR.

The potent but non-specific inhibitors of Pl3 K, LY294002 and wortmannin, also have been shown to inhibit the kinase function of mTOR but act through targeting the catalytic domain of the protein (ref. 21). Further to the inhibition of mTOR function by small molecules targeted to the kinase domain, it has been demonstrated that kinase dead mTOR cannot transmit the upstream activating signals to the downstream effectors of mTOR, PHAS-1 or p70S6 kinase (ref. 22). It is also shown that not all functions of mTOR are rapamycin sensitive and this may be related to the observation that rapamycin alters the substrate profile of mTOR rather than inhibiting its activity per se (ref. 23). Therefore, it is proposed that a kinase domain directed inhibitor of mTOR may be a more effective inhibitor of mTOR.

In addition to rapamycin's ability to induce growth inhibition (cytostasis) in its own right, rapamycin and its derivatives have been shown to potentiate the cytotoxicity of a number of chemotherapies including cisplatin, camptothecin and doxorubicin (reviewed in ref. 20). Potentiation of ionising radiation induced cell killing has also been observed following inhibition of mTOR (ref. 24) Experimental and clinical evidence has shown that rapamycin analogues are showing evidence of efficacy in treating cancer, either alone or in combination with other therapies (see refs. 10,18,20).

The vast majority of mTOR pharmacology to date has focused on inhibition of mTOR via rapamycin or its analogues. However, as noted above, the only non-rapamycin agents that have been reported to inhibit mTOR's activity via a kinase domain targetted mechanism are the small molecule LY294002 and the natural product wortmannin (ref. 21).

### SUMMARY OF THE INVENTION

The present inventors have identified compounds which are ATP-competitive inhibitors of mTOR, and hence are non-rapamycin like in their mechanism of action.

Accordingly, the first aspect of the present invention provides a compound of formula I: 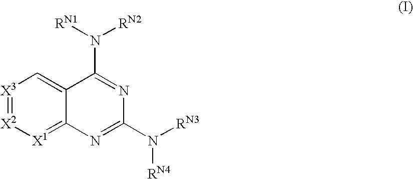
and isomers, salts, solvates, chemically protected forms, and prodrugs thereof, wherein:

one of X¹, X², and X³ is N, and the others are CH;

R^{N1} and R^{N2}, together with the nitrogen atom to which they are attached form a nitrogen-containing heterocyclic ring having from 4 to 8 ring atoms;

R^{N3} and R^{N4} together with the nitrogen atom to which they are attached form a nitrogen-containing heterocyclic ring having from 4 to 8 ring atoms.

A second aspect of the present invention provides a pharmaceutical composition comprising a compound of the first aspect and a pharmaceutically acceptable carrier or diluent.

A third aspect of the present invention provides a compound of the first aspect for use in a method of treatment of the human or animal body.

A fourth aspect of the present invention provides the use of a compound of formula 11: 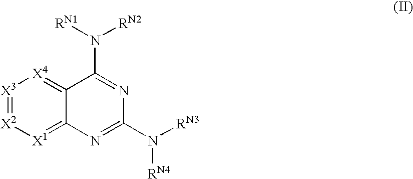
and isomers, salts, solvates, chemically protected forms, and prodrugs thereof in the preparation of a medicament for treating a disease ameliorated by the inhibition of mTOR, wherein:

one of X¹, X², X³ and X⁴ is N, and the others are CH

R^{N1} and R^{N2}, together with the nitrogen atom to which they are attached form a nitrogen-containing heterocyclic ring having from 4 to 8 ring atoms;

R^{N3} and R^{N4} together with the nitrogen atom to which they are attached form a nitrogen-containing heterocyclic ring having from 4 to 8 ring atoms.

Further aspects of the invention provide the use of a compound as defined in the fourth aspect of the invention in the preparation of a medicament for the treatment of: cancer, immuno-suppression, immune tolerance, autoimmune disease, inflammation, bone loss, bowel disorders, hepatic fibrosis, hepatic necrosis, rheumatoid arthritis, restinosis, cardiac allograft vasculopathy, psoriasis, beta-thalassaemia, and ocular conditions such as dry eye. mTOR inhibitors may also be effective as antifungal agents

Another further aspect of the invention provides for the use of a compound as defined in the fourth aspect of the invention in the preparation of a medicament for use as an adjunct in cancer therapy or for potentiating tumour cells for treatment with ionizing radiation or chemotherapeutic agents.

Other further aspects of the invention provide for the treatment of disease ameliorated by the inhibition of mTOR, comprising administering to a subject in need of treatment a therapeutically-effective amount of a compound as defined in the fourth aspect, preferably in the form of a pharmaceutical composition and the treatment of cancer, comprising administering to a subject in need of treatment a therapeutically-effective amount of a compound as defined in the fourth aspect in combination, preferably in the form of a pharmaceutical composition, simultaneously or sequentially with ionizing radiation or chemotherapeutic agents.

Definitions

Nitrogen-containing heterocyclic ring having from 4 to 8 ring atoms: The term “Nitrogen-containing heterocyclic ring having from 4 to 8 ring atoms” as used herein refers to a 4 to 8 membered heterocylic ring containing at least one nitrogen ring atom. Examples of these groups include, but are not limited to:

N₁: azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

N₁O₁S₁: oxathiazine (C₆).

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —C≡CH) and 2-propynyl (propargyl, —CH₂—C≡CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

Examples of cycloalkyl groups include, but are not limited to, those derived from:

saturated monocyclic hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C6), methylcyclopentane (C₆), dimethylcyclopentane (C₇), methylcyclohexane (C₇), dimethylcyclohexane (C₈), menthane (C₁₀);

unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);

saturated polycyclic hydrocarbon compounds:

thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);

unsaturated polycyclic hydrocarbon compounds:

camphene (C₁₀), limonene (C₁₀), pinene (C₁₀);

polycyclic hydrocarbon compounds having an aromatic ring:

indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Spiro-C₃₋₇ cycloalkyl or heterocyclyl: The term “spiro C₃₋₇ cycloalkyl or heterocyclyl” as used herein, refers to a C₃₋₇ cycloalkyl or C₃₋₇ heterocyclyl ring joined to another ring by a single atom common to both rings.

C₅₋₂₀ aryl: The term “C₅₋₂₀ aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅_20 carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C16).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₄ heteroaryl groups derived from acridine and xanthene.

The above alkyl, heterocyclyl, and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The cylic amino groups may be substituted on their ring by any of the substituents defined here, for example carboxy, carboxylate and amido.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇ alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group.

Examples of acylamido groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

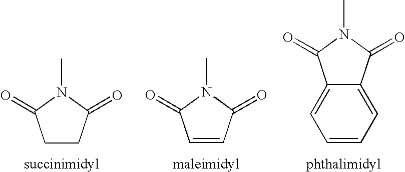

Ureido: —N(R¹)CONR²R³ wherein R² and R³ are independently amino substituents, as defined for amino groups, and R1 is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHC(═O)NHPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol : —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl).

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

As mentioned above, the groups that form the above listed substituent groups, e.g. C₁₋₇ alkyl, C₃₋₂₀ heterocyclyl and C₅₋₂₀ aryl, may themselves be substituted. Thus, the above definitions cover substituent groups which are substituted.

Further Preferences

The following preferences can apply to each aspect of the present invention, where applicable. The preferences for each group may be combined with those for any or all of the other groups, as appropriate.

X¹, X², X³ and X⁴

Preferably one of X¹, X² and X⁴ (where present) is N, and more preferably one of X¹ and X² is N. It is most preferred that X¹ is N.

R^{N1} and R^{N2}

R^{N1} and R^{N2}, together with the nitrogen atom to which they are attached, preferably form a nitrogen-containing heterocyclic ring having from 5 to 7 ring atoms. Preferred optionally substituted groups include, but are not limited, to morpholino, thiomorpholino, piperadinyl, piperazinyl (preferably N-substituted), homopiperazinyl (preferably N-substituted) and pyrrolidinyl. An additional preferred optionally substituted group is oxazepanyl.

Preferred N-substituents for the piperazinyl and homopiperazinyl groups include esters, in particular, esters bearing a C₁₋₇ alkyl group as an ester substituent, e.g. —C(═O)OCH₃, —C(═O)OCH₂CH₃ and —C(═O)OC(CH₃)₃.

More preferred groups are morpholino and pyrrolidinyl, with morpholino being the most preferred. These groups are preferably unsubstituted. In some embodiments, they may be substituted by one or more C₁₋₄ alkyl groups (e.g. methyl). A preferred group may be (3-methyl-morpholin-4-yl).

R^{N3} and R^{N4}

R^{N3} and R^{N4} preferably, together with the nitrogen atom to which they are attached, form a nitrogen-containing heterocyclic ring having from 5 to 7 ring atoms. Preferred optionally substituted groups include, but are not limited, to morpholino, thiomorpholino, piperadinyl, piperazinyl (preferably N-substituted), homopiperazinyl (preferably N-substituted) and pyrrolidinyl.

Preferred substituents for the groups include C₁₋₇ alkyl (e.g. methyl), amido (e.g. —C(═O)NH₂), hydroxy, ether, amino and esters, of which methyl, —C(═O)NH₂ and hydroxy are more preferred. The groups may bear 1, 2 or more substituents and these substituents may be in any position.

Preferred N-substituents for the piperazinyl and homopiperazinyl groups include esters, in particular, esters bearing a C₁₋₇ alkyl group as an ester substituent, e.g. —C(═O)OCH₃, —C(═O)OCH₂CH₃ and —C(═O)OC(CH₃)₃.

More preferred groups are morpholino (e.g. 3,5-dimethyl-morpholino) and piperadinyl (e.g. 4-amido-piperadinyl, 2-methyl-piperadinyl, 4-hydroxy-piperadinyl).

A particularly preferred set of groups are those defined by formula III: 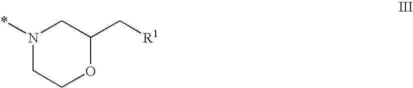
wherein R¹ is either:

(i) NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl, or together with the nitrogen atom to which they are attached form a nitrogen-containing heterocyclic ring having from 4 to 8 ring atoms; or(ii) OR^{O1}, where R^{O1} is selected from the group consisting of optionally substituted C₁₋₇ alkyl, optionally substituted C₃₋₂₀ heterocyclyl and optionally substituted C₅₋₂₀ aryl.

R^{N5} and R^{N6} may have the same preferences as R^{N3} and R^{N4}, except for being another group of formula II.

R^{O1} is preferably selected from optionally substituted C₅₋₂₀ aryl.

Particularly preferred compounds are shown in the examples. Other compounds of interest may include: 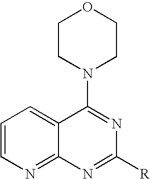
where R is selected from:

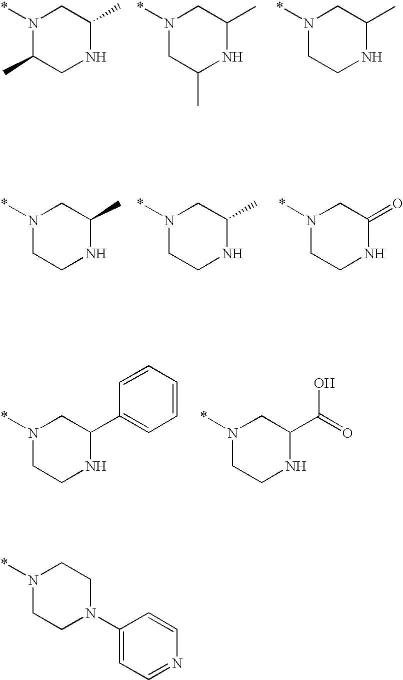

Includes Other Forms

Included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and /-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below, as well as its different polymorphic forms.

It may be convenient or desirable to prepare, purify, and/or handle a corresponding salt of the active compound, for example, a pharmaceutically-acceptable salt. Examples of pharmaceutically acceptable salts are discussed in ref. 25.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, ref. 26.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO.).

For example, a carboxylic acid group may be protected as an ester for example, as: an C₁₋₇ alkyl ester (e.g. a methyl ester; a t-butyl ester); a C₁₋₇ haloalkyl ester (e.g. a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₂₀ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O—P(═O)(OH)₂. Such a group can be cleared by phosphotase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

General Synthesis

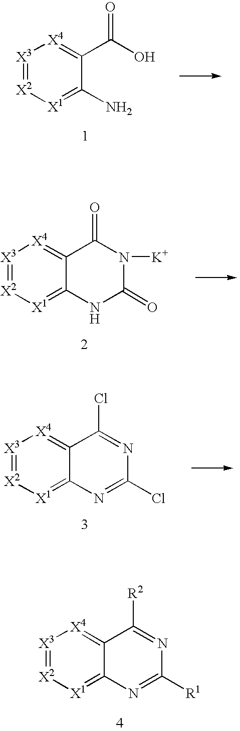

Compounds of formulae I and 11 can be represented by Formula I: 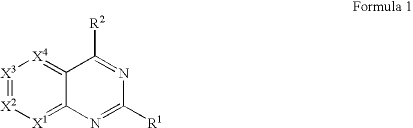
wherein in compounds of formula I, X⁴═CH, R¹ represents NR^{N3}R^{N4} and R² represents NR^{N1}R^{N2}. Compounds of Formula 1 can be synthesized from compounds of Formula 2: 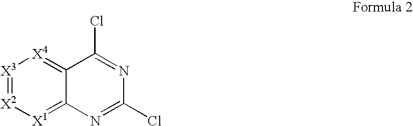
by reaction with HNR^{N1}R^{N2} (HR²) followed by reaction with HNR^{N3}R^{N4} (HR¹).

Compounds of Formula 2 can be synthesised from compounds of Formula 3: 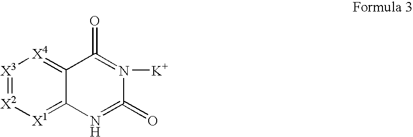
by treatment with POCl₃ and N,N-diiospropylamine, for example.

Compounds of Formula 3 can be synthesized from compounds of Formula 4: 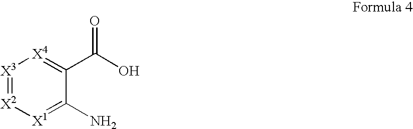
by treatment with potassium cyanate and ammonium chloride, for example.

Use

The present invention provides active compounds, specifically, active in inhibiting the activity of mTOR.

The term “active” as used herein, pertains to compounds which are capable of inhibiting mTOR activity, and specifically includes both compounds with intrinsic activity (drugs) as well as prodrugs of such compounds, which prodrugs may themselves exhibit little or no intrinsic activity.

One assay which may conveniently be used in order to assess the mTOR inhibition offered by a particular compound is described in the examples below.

The present invention further provides a method of inhibiting the activity of mTOR in a cell, comprising contacting said cell with an effective amount of an active compound, preferably in the form of a pharmaceutically acceptable composition. Such a method may be practised in vitro or in vivo.

For example, a sample of cells may be grown in vitro and an active compound brought into contact with said cells, and the effect of the compound on those cells observed. As examples of “effect”, the inhibition of cellular growth in a certain time or the accumulation of cells in the G1 phase of the cell cycle over a certain time may be determined. Where the active compound is found to exert an influence on the cells, this may be used as a prognostic or diagnostic marker of the efficacy of the compound in methods of treating a patient carrying cells of the same cellular type.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “adjunct” as used herein relates to the use of active compounds in conjunction with known therapeutic means. Such means include cytotoxic regimes of drugs and/or ionising radiation as used in the treatment of different cancer types. Examples of adjunct anti-cancer agents that could be combined with compounds from the invention include, but are not limited to, the following: alkylating agents: nitrogen mustards, mechlorethamine, cyclophosphamide, ifosfamide, melphalan, chlorambucil: Nitrosoureas: carmustine (BCNU), lomustine (CCNU), semustine (methyl-CCNU), ethylenimine/methylmelamine, thriethylenemelamine (TEM), triethylene thiophosphoramide (thiotepa), hexamethylmelamine (HMM, altretamine): Alkyl sufonates; busulfan; Triazines, dacarbazine (DTIC): Antimetabolites; folic acid analogs, methotrexate, trimetrexate, pyrimidine analogs, 5-fluorouracil, fluorodeoxyuridine, gemcitabine, cytosine arabinoside (AraC, cytarabine), 5-azacytidine, 2,2′-difluorodeoxycytidine: Purine analogs; 6-mercaptopurine, 6-thioguanine, azathioprine, 2′-deoxycoformycin (pentostatin, erythrohydroxynonyladenine (EHNA), fludarabine phosphate, 2-Chlorodeoxyadenosine (cladribine, 2-CdA): Topoisomerase I inhibitors; camptothecin, topotecan, irinotecan, rubitecan: Natural products; antimitotic drugs, paclitaxel, vinca alkaloids, vinblastine (VLB), vincristine, vinorelbine, Taxotere™ (docetaxel), estramustine, estramustine phosphate; epipodophylotoxins, etoposide, teniposide: Antibiotics; actimomycin D, daunomycin (rubidomycin), doxorubicin (adriamycin), mitoxantrone, idarubicin, bleomycins, plicamycin (mithramycin), mitomycin C, dactinomycin: Enzymes; L-asparaginase, RNAse A: Biological response modifiers; interferon-alpha, IL-2, G-CSF, GM-CSF: Differentiation Agents; retinoic acid derivatives: Radiosensitizers;, metronidazole, misonidazole, desmethylmisonidazole, pimonidazole, etanidazole, nimorazole, RSU 1069, E09, RB 6145, SR4233, nicotinamide, 5-bromodeozyuridine, 5-iododeoxyuridine, bromodeoxycytidine: Platinium coordination complexes; cisplatin, carboplatin: Anthracenedione; mitoxantrone, AQ4N Substituted urea, hydroxyurea; Methylhydrazine derivatives, N-methylhydrazine (MIH), procarbazine; Adrenocortical suppressant, mitotane (o.p′-DDD), aminoglutethimide: Cytokines; interferon (α, β, γ), interleukin; Hormones and antagonists; adrenocorticosteroids/antagonists, prednisone and equivalents, dexamethasone, aminoglutethimide; Progestins, hydroxyprogesterone caproate, medroxyprogesterone acetate, megestrol acetate; Estrogens, diethylstilbestrol, ethynyl estradiol/equivalents; Antiestrogen, tamoxifen; Androgens, testosterone propionate, fluoxymesterone/equivalents; Antiandrogens, flutamide, gonadotropin-releasing hormone analogs, leuprolide; Nonsteroidal antiandrogens, flutamide; EGFR inhibitors, VEGF inhibitors; Proteasome inhibitors.

Active compounds may also be used as cell culture additives to inhibit mTOR, for example, in order to sensitize cells to known chemotherapeutic agents or ionising radiation treatments in vitro.

Active compounds may also be used as part of an in vitro assay, for example, in order to determine whether a candidate host is likely to benefit from treatment with the compound in question.

Cancer

The present invention provides active compounds which are anticancer agents or adjuncts for treating cancer. One of ordinary skill in the art is readily able to determine whether or not a candidate compound treats a cancerous condition for any particular cell type, either alone or in combination.

Examples of cancers include, but are not limited to, lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma and leukemias.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutang, gibbon), or a human.

Formulations

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, refs. 27 to 29.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant,.such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml.

The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### EXAMPLES

General Experimental Methods

Thin Layer chromatography was carried out using Merck Kieselgel 60 F₂₅₄ glass backed plates. The plates were visualized by the use of a UV lamp (254 nm). Silica gel 60 (particle sizes 40-63 μm) supplied by E. M. Merck was employed for flash chromatography. ¹H NMR spectra were recorded at 300 MHz on a Bruker DPX-300 instrument. Chemical shifts were referenced relative to tetramethylsilane.

Purification and Identification of Library Samples

The samples were purified on Gilson LC units. Mobile phase A—0.1% aqueous TFA, mobile phase B—Acetonitrile; flow rate 6 ml/min; Gradient—typically starting at 90% A/10% B for 1 minute, rising to 97% after 15 minutes, holding for 2 minutes, then back to the starting conditions. Column: Jones Chromatography Genesis 4 μm, C18 column, 10 mm×250 mm. Peak acquisition based on UV detection at 254 nm.

Mass spectra were recorded on a Finnegan LCQ instrument in positive ion mode. Mobile phase A—0.1% aqueous formic acid. Mobile phase B—Acetonitrile; Flowrate 2 ml/min; Gradient—starting at 95% A/5% B for 1 minute, rising to 98% B after 5 minutes and holding for 3 minutes before returning to the starting conditions. Column: Varies, but always C18 50 mm×4.6 mm (currently Genesis C18 4 μm. Jones Chromatography). PDA detection Waters 996, scan range 210-400 nm.

Microwave Synthesis

Reactions were carried out using a Personal Chemistry™ Emrys Optimiser microwave synthesis unit with robotic arm. Power range between. 0-300 W at 2.45 GHz. Pressure range between 0-20 bar; temperature increase between 2-5° C./sec; temp range 60-250° C.

### Example 1

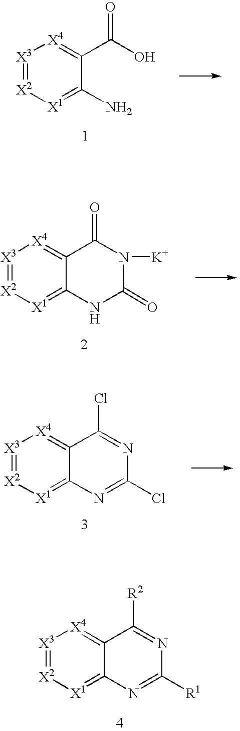

Starting Materials:

1a: X¹═N, X²═CH, X³═CH, X⁴═CH: 2-amino-nicotinic acid

1b: X¹═CH, X²═CH, X³═N, X⁴═CH: 3-amino-isonicotinic acid

1c: X¹═CH, X²═CH, X³═CH, X⁴═N: 3-Amino-pyridine-2-carboxylic acid

### (i) 1H-Pyridopyrimidine-2, 4-diones (2)

The appropriate amino acid (1)(1 equivalent), potassium cyanate (5 equivalents) and ammonium chloride (10 equivalents) were suspended in water. The slurry was heated (160° C.) and mixed manually for 2 hours as water vapour was expelled from the reaction vessel. The reaction temperature was then raised to 200° C. for 40 minutes before being cooled to 90° C. whereupon hot water was added and then the mixture was allowed to cool to room temperature. The precipitate that formed during cooling was removed by filtration, washed with water (twice), and diethyl ether (once) before being dried in a desiccator to give the desired product in suitably clean form to be used without further purification.

2a: 1H-Pyrido[2,3-d]pyrimidine-2,4-dione: m/z (LC-MS, ESP): does not ionise R/T=0.76 mins

2b: 1H-Pyrido[3,4-d]pyrimidine-2,4-dione: m/z (LC-MS, ESP): 164 [M-K+H]+, R/T=0.38 mins

2c: 1H-Pyrido[3,2-d]pyrimidine-2,4-dione: m/z (LC-MS, ESP): 164 [M-K+H]+,R/T=0.45 mins

### (ii) 2,4-Dichloro-pyridopyrimidines (3)

The appropriate 1H-pyridopyrimidine-2,4-dione (2)(1 equivalent) was dissolved in POCl₃ (44 equivalents). To this mixture was added N,N-diisopropylamine (2.8 equivalents) in a dropwise fashion. The reaction was stirred at room temperature under an inert atmosphere for 5 hours. After this time the mixture was concentrated in vacuo while taking care to keep the temperature below 30° C. The resulting black residue was poured onto crushed ice. The mixture was extracted with CH₂Cl₂ (×2) and the organic extracts then washed with water, dried (MgSO₄), filtered and concentrated in vacuo to provide a tar like material that corresponded to the desired product in suitably clean form to be used without any further purification.

3a: 2,4-Dichloro-pyrido[2,3-d]pyrimidine: m/z (LC-MS, ESP): 200 [M+H]⁺R/T=3.60 mins

3b: 2,4-Dichloro-pyrido[3,4-d]pyrimidine: m/z (LC-MS, ESP): 200 [M+H]⁺, R/T=3.82 mins

3c: 2,4-Dichloro-pyrido[3,2-d]pyrimidine: m/z (LC-MS, ESP): 200 [M+H]⁺,R/T=3.80 mins

### (iii) Pyridopyrimidine-2,4-diamines (4)

The appropriate 2,4-dichloro-pyridopyrimidine (3)(1 equivalent) was suspended in CH₂Cl₂ (4 ml of solvent per mmol of material) and to this mixture was added triethylamine (1 equivalent). The resultant orange solution was then cooled to 0° C. and the appropriate amine (R²H) (1 equivalent) added dropwise as a 0.1 M solution in CH₂Cl₂ over 5 minutes. The mixture was stirred for a further 45 minutes before it was diluted with water and extracted with CH₂Cl₂ (×2). The organic extracts were dried using MgSO₄, filtered and concentrated in vacuo to give a crude solid that was purified by flash chromatography (SiO₂) using Hexanes:EtOAc (2:3) as eluent to give the desired product (1 equivalent) which was diluted in dimethylacetamide (0.7 M) and the appropriate amine (R¹H) (2.5 equivalents) added. The reaction mixture was heated to 60° C. for 16 hours. Upon completion the reaction mixture was submitted for preparative HPLC purification to give the desired pyridopyrimidine-2,4-diamines, as detailed below:

| | | | | | | | Purity | m/z | RT |
|---|---|---|---|---|---|---|---|---|---|
| | X¹ | X² | X³ | X⁴ | R¹ | R² | % | [M + H]⁺ | (mins) |
| 4a | N | CH | CH | CH | 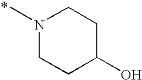 | 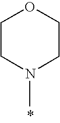 | 85 | 317 | 2.51 |
| 4b | N | CH | CH | CH | 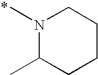 | 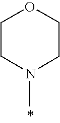 | 99 | 314 | 3.11 |
| 4c | N | CH | CH | CH | 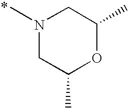 | 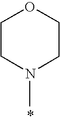 | 90 | 330 | 2.97 |
| 4d | CH | N | CH | CH | 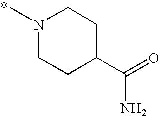 | 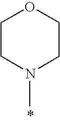 | — | — | — |
| 4e | CH | CH | CH | N | 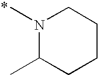 | 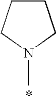 | 100 | 416 | 3.4 |
| 4f | N | CH | CH | CH | 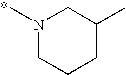 | 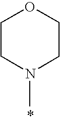 | 96.0 | 314.2 | 3.92 |
| g | N | CH | CH | CH | 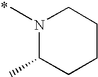 | 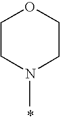 | 100.0 | 314.3 | 3.95 |
| 4h | N | CH | CH | CH | 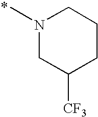 | 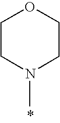 | 100.0 | 368.2 | 4.18 |
| 4i | N | CH | CH | CH | 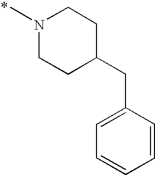 | 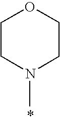 | 100.0 | 390.3 | 4.60 |
| 4j | N | CH | CH | CH | 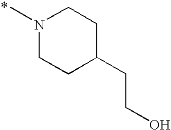 | 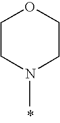 | 100.0 | 344.3 | 3.60 |
| 4k | N | CH | CH | CH | 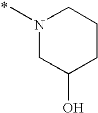 | 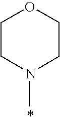 | 100.0 | 316.2 | 3.43 |
| 4l | N | CH | CH | CH | 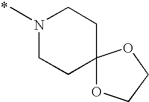 | 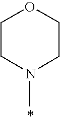 | 98.0 | 358.2 | 3.70 |
| 4m | N | CH | CH | CH | 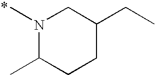 | 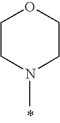 | 98.0 | 330.2 | 3.67 |
| 4n | N | CH | CH | CH | 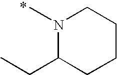 | 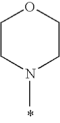 | 91.0 | 328.3 | 4.22 |
| 4o | N | CH | CH | CH | 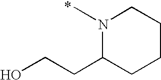 | 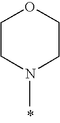 | 99.0 | 344.3 | 3.70 |
| 4p | N | CH | CH | CH | 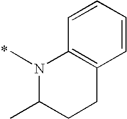 | 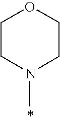 | 91.0 | 362.2 | 4.30 |
| 4q | N | CH | CH | CH | 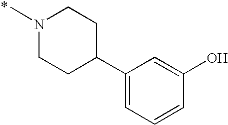 | 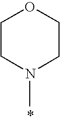 | 100.0 | 392.3 | 4.05 |
| 4r | N | CH | CH | CH | 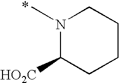 | 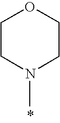 | 91.0 | 344.2 | 3.70 |
| 4s | N | CH | CH | CH | 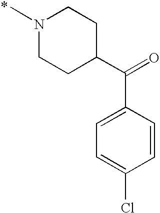 | 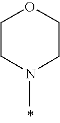 | 100.0 | 438.2 | 4.56 |
| 4t | N | CH | CH | CH | 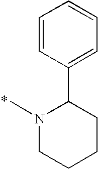 | 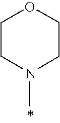 | 96.0 | 376.2 | 4.22 |
| 4u | N | CH | CH | CH | 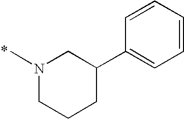 | 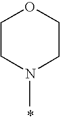 | 99.0 | 376.2 | 4.24 |
| 4v | N | CH | CH | CH | 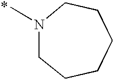 | 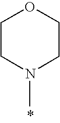 | 100.0 | 314.5 | 3.16 |
| 4w | N | CH | CH | CH | 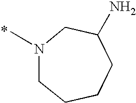 | 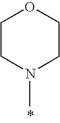 | 85.0 | 328.5 | 3.30 |
| 4x | N | CH | CH | CH | 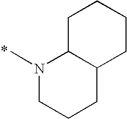 | 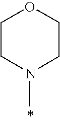 | 99.0 | 354.5 | 3.58 |
| 4y | N | CH | CH | CH | 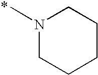 | 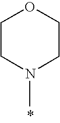 | 99.0 | 300.4 | 2.98 |
| 4z | N | CH | CH | CH | 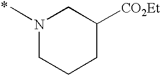 | 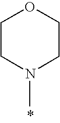 | 85.0 | 372.4 | 3.18 |
| 4aa | N | CH | CH | CH | 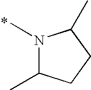 | 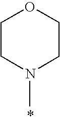 | 100.0 | 314.3 | 3.90 |
| 4ab | N | CH | CH | CH | 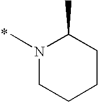 | 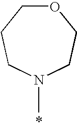 | 100.0 | 328.3 | 3.94 |
| 4ac | N | CH | CH | CH | 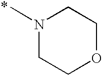 | 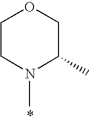 | 100.0 | 316.0 | 3.34 |
| 4ad | N | CH | CH | CH | 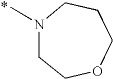 | 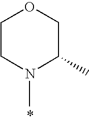 | 100.0 | 330.1 | 3.38 |
| 4ae | N | CH | CH | CH | 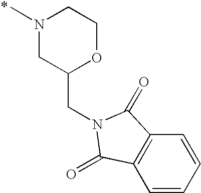 | 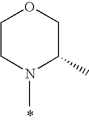 | 93.3 | 474.9 | 7.30 |
| 4af | N | CH | CH | CH | 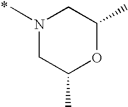 | 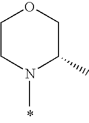 | 100.0 | 344.1 | 3.62 |
| 4ag | N | CH | CH | CH | 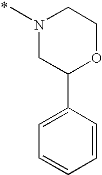 | 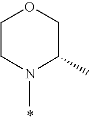 | 100.0 | 392.1 | 4.09 |
| 4ah | N | CH | CH | CH | 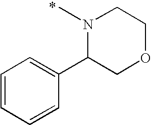 | 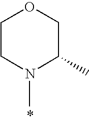 | 95.0 | 392.0 | 7.17 |
| 4ai | N | CH | CH | CH | 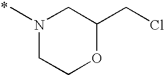 | 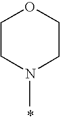 | 99.0 | 350.3 | 3.57 |

### Example 2

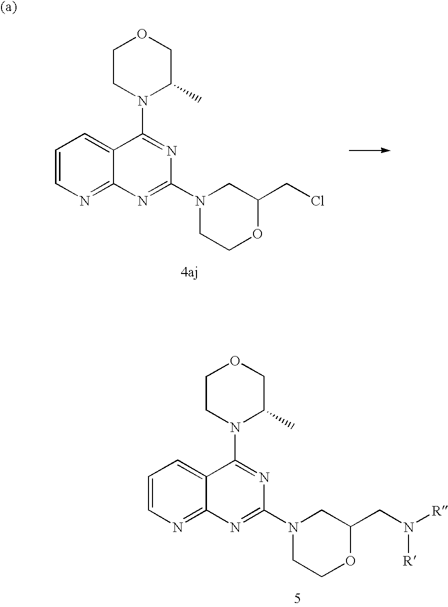

To a solution of 2-(2-Chloromethyl-morpholin-4-yl)-4-((S)-3-methyl-morpholin-4-yl)-pyrido[2,3-d]pyrimidine (4aj)(36 mg, 0.1 mmol) and the appropriate amine (0.5 mmol) in dimethylacetamide (2.5 ml) was added Nal (3 mg, 0.02 mmol) and K₂CO₃ (14 mg, 0.1 mmol). The reaction vessel was sealed and heated under the influence of microwave radiation (low absorption setting, 200° C., 20 minutes). After this the crude reaction mixture was concentrated in vacuo and purified by preparative HPLC to give the desired compounds (5).

| | | Purity | m/z | RT |
|---|---|---|---|---|
| | NR′R″ | % | [M + H]⁺ | (mins) |
| 5a | 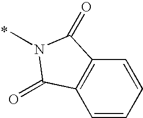 | 100.0 | 461.3 | 3.91 |
| 5b | 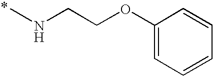 | 99.0 | 451.4 | 3.43 |
| 5c | 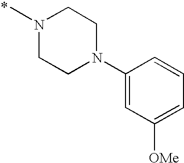 | 87.1 | 520.6 | 3.54 |
| 5d | 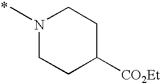 | 96.0 | 485.5 | 3.31 |
| 5e | 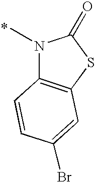 | 97.0 | 513.5 | 4.81 |
| 5f | 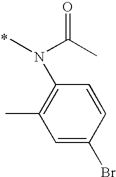 | 95.0 | 559.3 | 4.65 |
| 5g | 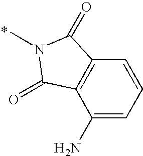 | 96.3 | 490.5 | 4.05 |
| 5h | 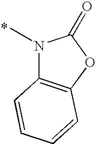 | 96.9 | 463.5 | 4.17 |
| 5i | 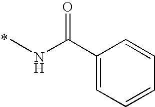 | 99.3 | 449.4 | 3.82 |
| 5j | 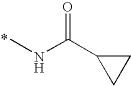 | 99.2 | 413.5 | 3.54 |
| 5k | 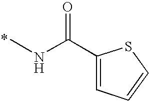 | 98.3 | 455.5 | 3.76 |
| 5l | 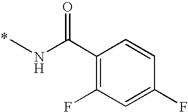 | 98.8 | 485.5 | 3.97 |
| 5m | 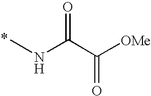 | 83.1 | 431.4 | 3.48 |
| 5n | 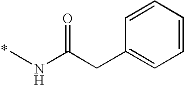 | 98.0 | 463.4 | 3.84 |
| 5o | 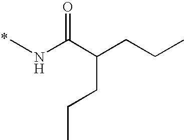 | 100.0 | 471.5 | 4.20 |
| 5p | 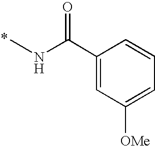 | 479.4 | 99.4 | 3.89 |

(b)

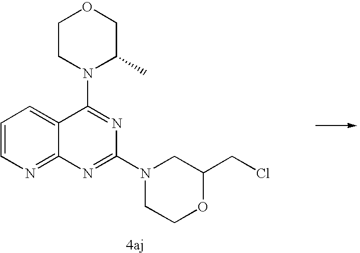

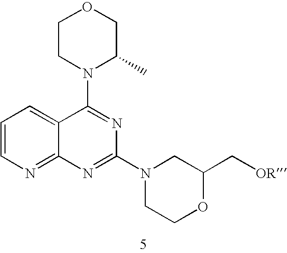

To a solution of 2-(2-Chloromethyl-morpholin-4-yl)-4-((S)-3-methyl-morpholin-4-yl)-pyrido[2,3-d]pyrimidine (4aj)(36 mg, 0.1 mmol) (11 mg, 0.03 mmol) in anhydrous dimethylacetamide (0.5 ml) was added tert-BuOK (6.8 mg, 0.6 mmol), and 18-crown-6 ether (0.006 mmol, 1.6 mg). The appropriate alcohol was then added to the reaction mixture and each reaction heated to 110° C. for 15 hours. After this the crude reaction mixture was concentrated in vacuo and purified by preparative HPLC to give the desired compounds (5).

| | | Purity | m/z | RT |
|---|---|---|---|---|
| | OR′″ | % | [M + H]⁺ | (mins) |
| 5r | 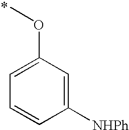 | 97.0 | 513.5 | 4.81 |
| 5s | 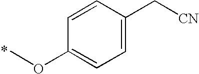 | 92.0 | 461.2 | 4.26 |
| 5t | 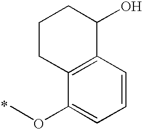 | 98.0 | 492.1 | 4.18 |
| 5u | 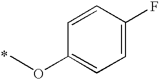 | 78.0 | 440.1 | 4.46 |
| 5v | 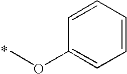 | 100.0 | 422.2 | 4.40 |
| 5w | 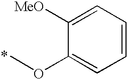 | 96.0 | 452.1 | 4.32 |
| 5x | 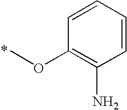 | 91.0 | 437.1 | 3.69 |
| 5y | 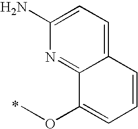 | 93.0 | 488.1 | 3.58 |
| 5z | 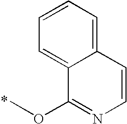 | 98.0 | 473.1 | 4.17 |
| 5aa | 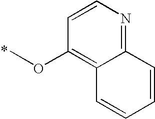 | 90.0 | 473.1 | 6.30 |
| 5ab | 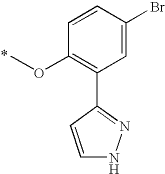 | 100.0 | 568.0 | 4.68 |
| 5ac | 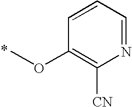 | 78.0 | 448.1 | 4.04 |
| 5ad | 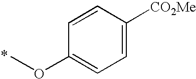 | 82.9 | 480.1 | 4.39 |
| 5ae | 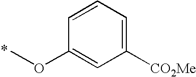 | 69.0 | 480.1 | 4.43 |
| 5af | 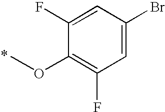 | 95.4 | 436.0 | 4.85 |

### Example 3

### Biological Assay

For mTOR enzyme activity assays, mTOR protein was isolated from HeLa cell cytoplasmic extract by immunoprecipitation, and activity determined essentially as described previously using recombinant PHAS-1 as a substrate (ref. 21).

All the compounds tested exhibited IC₅₀ values less than 15 μM.

The following compounds exhibited IC₅₀ values less than 1.5 μM: 4c, 4d, 4h, 4n, 4o, 4y, 4ab, 4af, 4ag, 4ah, 5e, 5g, 5h, 5k, 5m, 5z.

### REFERENCE LIST

The following documents are all herein incorporated by reference.
1) Brown, et al., Nature, 369, 756-758 (1994)
2) Chiu, et al., Proc Natl Acad Sci, 91,12574-12578 (1994)
3) Sabatini, et al., Cell, 78, 35-43, (1994)
4) Sabers, et al., J Biol Chem, 270, 825-822 (1995)
5) Abraham, Curr Opin Immunol, 8, 412-418 (1996)
6) Schmelze and Hall, Cell, 103, 253-262 (2000)
7) Burnett, et al., Proc Natl Acad Sci, 95, 1432-1437 (1998)
8) Terada, et al., Proc Natl Acad Sci, 91, 11477-11481 (1994)
9) Jeffries, et al., EMBO J, 16, 3693-3704 (1997)
10) Bjornsti and Houghton, Nat Rev Cancer, 4, 335-348 (2004)
11) Gingras, et al., Genes Dev, 13, 1422-1437 (1999)
12) Gingras, et al., Genes Dev, 15, 807-826 (2001)
13) Neuhaus, et al., Liver Transplantation, 7, 473-484 (2001)
14) Woods and Marks, Ann Rev Med, 55, 169-178 (2004)
15) Dahia, Endocrine-Related Cancer, 7, 115-129 (2000)
16) Cristofano and Pandolfi, Cell, 100, 387-390 (2000)
17) Samuels, et al., Science, 304, 554 (2004)
18) Huang and Houghton, Curr Opin Pharmacol, 3, 371-377 (2003)
19) Sawyers, Cancer Cell, 4, 343-348 (2003)
20) Huang and Houghton, Curr Opin in Invest Drugs, 3, 295-304 (2002)
21) Brunn, et al., EMBO J, 15, 5256-5267 (1996)
22) Edinger, et al., Cancer Res, 63, 8451-8460, (2003)
23) Lawrence, et al., Curr Top Microbiol Immunol, 279, 199-213 (2004)
24) Eshleman, et al., Cancer Res, 62, 7291-7297 (2002)
25) Berge, et al., J. Pharm. Sci., 66, 1-19 (1977).
26) Green, T. and Wuts, P., “Protective Groups in Organic Synthesis”, 3rd Edition, John Wiley and Sons (1999)
27) “Handbook of Pharmaceutical Additives”, 2nd Edition (eds. M. Ash and 1. Ash), 2001 (Synapse Information Resources, Inc., Endicott, N.Y., USA).
28) “Remington's Pharmaceutical Sciences”, 20th edition, pub. Lippincott, Williams & Wilkins, 2000
29) “Handbook of Pharmaceutical Excipients”, 2nd edition, 1994.

1. A compound of formula I: 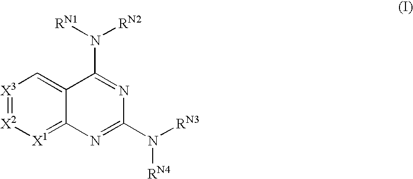
and isomers, salts, solvates, chemically protected forms, and prodrugs thereof one of X¹, X² and X³ is N, and the others are CH;
R^{N1} and R^{N2} together with the nitrogen atom to which they are attached form a nitrogen-containing heterocyclic ring having from 4 to 8 ring atoms;
R^{N3} and R^{N4} together with the nitrogen atom to which they are attached form a nitrogen-containing heterocyclic ring having from 4 to 8 ring atoms.

2. A compound according to claim 1, wherein one of X¹ and X² is N.

3. A compound according to claim 2, wherein X¹ is N.

4. A compound according to claim 1, wherein R^{N1} and R^{N2}, together with the nitrogen atom to which they are attached, form an optionally substituted group selected from morpholino, oxazenpenyl, thiomorpholino, piperadinyl, piperazinyl, homopiperazinyl and pyrrolidinyl.

5. A compound according to claim 4, wherein R^{N1} and R^{N2}, together with the nitrogen atom to which they are attached, form morpholino or 3-methyl-morpholin-4-yl.

6. A compound according to claim 1, wherein R^{N3} and R^{N4}, together with the nitrogen atom to which they are attached, form an optionally substituted group selected from morpholino, thiomorpholino, piperadinyl, piperazinyl, homopiperazinyl and pyrrolidinyl.

7. A compound according to claim 6, wherein R^{N3} and R^{N4}, together with the nitrogen atom to which they are attached, form an optionally substituted group selected from morpholino and piperadinyl.

8. A compound according to claim 7, wherein R^{N3} and R^{N4}, together with the nitrogen atom to which they are attached, form a group of formula III: 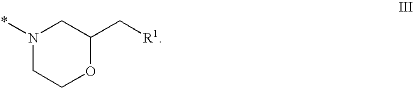
wherein R¹ is either:
(i) NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl, or together with the nitrogen atom to which they are attached form a nitrogen-containing heterocyclic ring having from 4 to 8 ring atoms; or
(ii) OR^{O1}, where R^{O1} is selected from the group consisting of optionally substituted C₁₋₇ alkyl, optionally substituted C₃₋₂₀ heterocyclyl and optionally substituted C₅₋₂₀ aryl.

9. A pharmaceutical composition comprising a compound of claim 1 and a pharmaceutically acceptable carrier or diluent.

10. A method of treatment of a disease ameliorated by the inhibition of mTOR, comprising administering to a subject in need of treatment a therapeutically-effective amount of a compound of formula II: 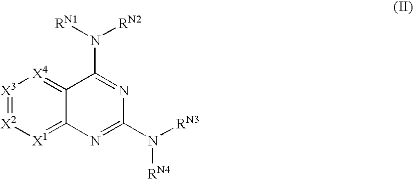
and isomers, salts, solvates, chemically protected forms, and prodrugs thereof, wherein:
one of X¹, X², X³ and X⁴ is N, and the others are CH;
R^{N1} and R^{N2} together with the nitrogen atom to which they are attached form a nitrogen-containing heterocyclic ring having from 4 to 8 ring atoms;
R^{N3} and R^{N4} together with the nitrogen atom to which they are attached form a nitrogen-containing heterocyclic ring having from 4 to 8 ring atoms.

11. The method according to claim 10, wherein X¹ is N.

12. The method according to claim 10, wherein R^{N1} and R^{N2}, together with the nitrogen atom to which they are attached, form morpholino or 3-methyl-morpholin-4-yl.

13. The method according to claim 10, wherein R^{N3} and R^{N4}, together with the nitrogen atom to which they are attached, form an optionally substituted group selected from morpholino and piperadinyl.

14. The method according to claim 13, wherein R^{N3} and R^{N4}, together with the nitrogen atom to which they are attached, form a group of formula III: 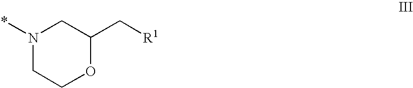
wherein R¹ is either:
(i) NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl, or together with the nitrogen atom to which they are attached form a nitrogen-containing heterocyclic ring having from 4 to 8 ring atoms; or
(ii) OR^{O1}, where R^{O1} is selected from the group consisting of optionally substituted C₁₋₇ alkyl, optionally substituted C₃₋₂₀ heterocyclyl and optionally substituted C₅₋₂₀ aryl.

15. The method according to claim 10, wherein the disease ameliorated by the inhibition of mTOR is selected from cancer, immuno-suppression, immune tolerance, autoimmune disease, inflammation, bone loss, bowel disorders, hepatic fibrosis, hepatic necrosis, rheumatoid arthritis, restinosis, cardiac allograft vasculopathy, psoriasis, beta-thalassaemia, and ocular conditions.

16. A method of treatment of cancer, comprising administering to a subject in need of treatment a therapeutically-effective amount of a compound of formula II: 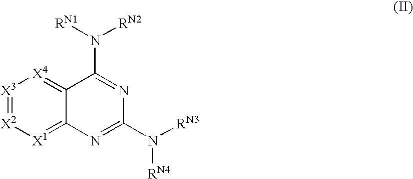
and isomers, salts, solvates, chemically protected forms, and prodrugs thereof, simultaneously or sequentially with ionizing radiation or chemotherapeutic ageints wherein:
one of X¹, X², X³ and X⁴ is N, and the others are CH;
R^{N1} and R^{N2} together with the nitrogen atom to which they are attached form a nitrogen-containing heterocyclic ring having from 4 to 8 ring atoms;
R^{N3} and R^{N4} together with the nitrogen atom to which they are attached form a nitrogen-containing heterocyclic ring having from 4 to 8 ring atoms.

17. The method according to claim 16, wherein X¹ is N.

18. The method according to claim 16, wherein R^{N1} and R^{N2}, together with the nitrogen atom to which they are attached, form morpholino or 3-methyl-morpholin-4-yl.

19. The method according to claim 16, wherein R^{N3} and R^{N4}, together with the nitrogen atom to which they are attached, form an optionally substituted group selected from morpholino and piperadinyl.

20. The method according to claim 16, wherein R^{N3} and R^{N4}, together with the nitrogen atom to which they are attached, form a group of formula III: 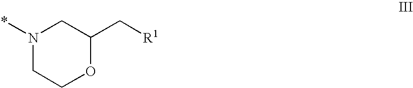
wherein R¹ is either:
(i) NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl, or together with the nitrogen atom to which they are attached form a nitrogen-containing heterocyclic ring having from 4 to 8 ring atoms; or
(ii) OR^{O1}, where R^{O1} is selected from the group consisting of optionally substituted C₁₋₇ alkyl, optionally substituted C₃₋₂₀ heterocyclyl and optionally substituted C₅₋₂₀ aryl.
